(19) United States
(12) Patent Application Publication
(10) Pub. No.: US 2007/0031815 A1
(43) Pub. Date: Feb. 8, 2007

(54) Screening method for identifying hsp90 modulators

(75) Inventors: John Jenkins, Liverpool (GB); William Greenhalf, Liverpool (GB); David Ian O'Connor, Staffordshire (GB)

Correspondence Address: DAVIS WRIGHT TREMAINE LLP, 865 FIGUEROA STREET, SUITE 2400, LOS ANGELES, CA, 90017-2566, US

(73) Assignee: University of Liverpool, Liverpool (GB)

(21) Appl. No.: 10/554,973

(22) PCT Filed: Apr. 28, 2004
(86) PCT No.: PCT/GB04/01828

§ 371 Date: Oct. 31, 2005

(30) Foreign Application Priority Data
May 1, 2003 (GB) 0310017.9

(51) Int. Cl. C12Q 1/68; C12Q 1/00; C12N 1/18

(52) U.S. Cl. 435/4; 435/6; 435/254.2

(57) ABSTRACT

A screening method for identifying and/or analysing Hsp90 inhibitors and/or Hsp90 agonists comprises the steps of contacting a compound with at least two of yeast strains A-E wherein each yeast strain comprises expression vectors from which a pair of binding partners for a yeast two-hybrid assay are expressed. The binding partner pairs comprise: A: Hsp90-targeting protein; B: Hsp90-Hsp90; C: Hsp90-p23; D: Hsp90-E3 ligase; E: Hsp90-Client. Inhibition and/or promotion of dimerisation between the binding partners is then measured.

The present invention relates to a screen for Hsp90 antagonists or agonists using reporters in yeast that are activated by specific interactions of Hsp90 with other proteins.

Hsp90 is a protein that is conserved from yeast to mammals. There are three structurally equivalent isoforms of Hsp90 in mammalian cells: An endoplasmic reticulum associated form (GRP94) and two predominantly cytoplasmic forms, Hsp90α (serum responsive expression) and Hsp90β (stress induced expression). There is also a mitochondrial form (TRAP-1) but this is structurally distinct from the other forms in that it lacks a central hinge region. The term “Hsp90” used herein will be used as a generic term referring only to the structurally equivalent isoforms.

Hsp90 is a multifunctional protein with target or client proteins (as defined hereafter) involved in signalling, the cell cycle and apoptosis. Specificity for clients is determined by co-chaperones (targeting proteins). Hsp90 has the following functions:
(a) It targets proteins which it holds in an inactive state.
(b) It targets proteins which it causes to be destroyed
(c) It targets proteins as a classical chaperone (i.e. folding the protein from a nascent state). The variant of Hsp90 involved in this is principally the GRP94 form, the best characterised target of GRP94 being the cystic fibrosis related chloride ion channel CFTR. It is believed that Hsp90 is involved in the targeted destruction of CFTR mutants as Hsp90 inhibition increases the stability of these mutant proteins.
(d) Another function of Hsp90 is the activation of other proteins by interactions at the TPR binding domain on the C-terminal of Hsp90, such proteins include the phosphatase PP5 and the mitochondrial import protein MAS70 (TOM70). A Tetratricopeptide Repeat (TPR) domain is a degenerate 34 amino acid consensus sequence that is believed to mediate protein-protein binding. Such domains are found in various Hsp90 cofactors, including Hop and the immunophilins, such as FKBP52 and CyP40. The targeting protein Cdc37 does not bind to the Hsp90 at the same site as the TPR domain proteins, but the binding site of Cdc37 is close to this site so that occupation of the Cdc37 site interferes with binding of large TPR proteins. Cdc37 and Hop1 also bind to the N-terminal of Hsp90. It is assumed that Cdc37 binds to the C-terminal of Hsp90 after first binding Client proteins, before this it binds to the N-terminal of Hsp90 preventing ATP binding.

Antagonists of Hsp90 are a class of powerful anti-microbial and anti-tumour agents. The best characterised family of Hsp90 antagonists is the benzoquinone ansamycins (e.g. Geldanainycin, Herbimycin and Macbecin I). Geldanamycin (GA) was first identified as the active agent which allowed the inhibitition of the growth of the protozoa Tetrahymena pyriformis by a strain of the actinomycete Streptomyces hygroscopicus. The activity of GA against T. pyriformis and other protozoa was found to be only moderate given the poor solubility of the antibiotic (Minimal Inhibitory Concentration, MIC=2 μg/ml). However, GA was more active against fungi (for Botrytis MIC <0.5 ng/ml) and a small market was achieved as an anti-fungal agent. Initial studies also indicated a very strong activity against mammalian cells (active at 0.1 ng/ml for KB cells). Despite these early reports its use as an anti-tumour agent was not discussed until it was identified during the NCI in-vitro screen for anti-tumour agents 25 years later, in 1995. GA itself was too toxic for therapeutic use but a derivative, 17-allylamino demethoxy geldanamycin (17-AAG), is completing phase I clinical trials in the USA (Memmorial Sloan Kettering Cancer Center and the NCI) and the UK (ICR, Sutton). The molecular target of GA was initially believed to be tyrosine-kinases, but crystallographic studies have shown that GA binds to an ATP binding site on the chaperone protein Hsp90. GA binding to Hsp90 depletes tyrosine kinases such as Src and ErbB2, hence the initial assumption that geldanamycin acts through these proteins. The drug also affects a range of other cancer related proteins including; Raf-1 and mt-p53.

Radicicol (RAD) is another antibiotic that was identified in an earlier screen for anticancer agents (a small scale screen for agents that could reverse the transformed phenotype of Rous sarcoma virus-transformed fibroblasts). As with GA the initial assumption was that RAD acted against tyrosine kinases. However it was later determined to bind to the same ATP binding site on Hsp90 as GA, despite the lack of any obvious structural similarity between the two compounds.

In addition to benzoquinone ansamycins and radicicol other antitumour agents have also been suggested to act via Hsp90 inhibition. These include cisplatin and novobiocin. Cisplatin is a well-established chemotherapeutic agent that acts as a DNA cross linker. It was identified in a rational screen of platinum compounds for cytotoxic properties. A connection with Hsp90 was first identified because of the specific induction of Hsp90 (rather than other chaperones such as Hsp70) in rats with cisplatin induced renal failure. Recently it was established that cisplatin binds with a high degree of specificity to the C-terminal of Hsp90 (distant from the ATP pocket) and causes ATP sensitive inhibition of chaperone activity in vitro. It is unlikely that cisplatin's anti-Hsp90 activity is of any particular clinical relevance but the coumarin antibiotic novobiocin was identified as having anti-cancer activity on the basis of the possibility that it would bind to Hsp90. Novobiocin binds to the ATP binding domain of bacterial DNA gyrase B. As the ATP binding domain of Hsp90 and gyrase B are very similar it was hypothesised that novobiocin should also bind to Hsp90. Novobiocin also caused reduction in the levels of several Hsp90 clients (e.g. Erb-B2, v-Src, Raf-1 and mt-p53). It was found however, that the binding site for novobiocin is not the N-terminal ATP binding site but rather is situated in the C-terminal region of Hsp90.

Hsp90 inhibitors therefore have clinical value, but identification of Hsp90 antagonists up until this point has been serendipitous. GA was identified due to its cytotoxic effects and not because it was suspected to interact with Hsp90. Derivatives of GA have been investigated empirically, and although these modifications have been discussed in terms of predicted interaction with Hsp90 these discussions have dealt exclusively with improvement of existing interactions and not with exploitation of new interactions. It will therefore be appreciated that there is a need in the art to provide a method for screening and identifying agents that inhibit Hsp90 and, depending upon the nature of the inhibition, may be clinically useful.

Known techniques for identifying agents that interact with Hsp90 involve inhibition of growth in micro-organisms or in cell lines generated from tumours. Compounds that inhibit growth are characterised and then tested for Hsp90 binding. This methodology has the disadvantage that it does not give any indication of the specificity of any of the putative Hsp90 antagonists. Many compounds exist which are cytotoxic but of no therapeutic benefit and the most clinically relevant Hsp90 antagonists (17-AAG) is only mildly cytotoxic and would probably have been missed by such a screen. Also this is a standard approach and is acknowledged to miss many clinically important compounds and identify compounds with unacceptable general toxicity.

More recently the ICR in Sutton (UK) (http://www.icr.ac.uk/cctherap/analvtical.htm) have instigated a screening programme for Hsp90 inhibitors based upon the observed upregulation of Hsp70 as a result of Hsp90 inhibition in cell lines. This could be put into a general class of screening systems, whereby any protein known to be upregulated or downregulated by Hsp90 inhibition is assayed, by ELISA or similar technique, to identify possible Hsp90 antagonists. This technique has the disadvantage of being non-specific, i.e. mechanisms other than Hsp90 inhibition may result in an up-regulation of Hsp90 related proteins (e.g. Hsp70) or downregulation of Hsp90 clients (Raf-1, Cdk4). It is also an expensive test and is restricted to identifying agents relevant to cancer therapy. Furthermore Hsp90 inhibition has uses outside of the area of cancer therapeutics (e.g. anti-fungal therapeutics).

It is an object of the present invention to provide a screen for identifying compounds that modulate Hsp90, and in particular to provide specific information about tested compounds that will indicate that the tested compound will have efficacy for specific clinical indications.

According to a first aspect of the invention there is provided a screening method for identifying and/or analysing Hsp90 inhibitors and/or Hsp90 agonists comprising the steps of contacting a compound with at least two of yeast strains A-E wherein each yeast strain comprises expression vectors from which a pair of binding partners for a yeast two-hybrid assay are expressed and wherein the binding partner pairs comprise:

A: Hsp90-targeting protein;

B: Hsp90-Hsp90;

C: Hsp90-p23;

D: Hsp90-E3 ligase;

E: Hsp90-Client;

and measuring inhibition and/or promotion of dimerisation between the binding partners.

According to a second aspect of the invention, there is provided use of an Hsp90 inhibitor or an Hsp90 agonist identified by the screening method according to the first aspect of the invention as a medicament.

The medicament may be used in cancer therapy, or chemotherapy, for example, as described in Examples 1 & 2, or as an antimicrobial agent, for example, as described in Example 3.

By “Hsp90” we mean any of the structurally similar isoforms (i.e. Hsp90 alpha, beta or Grp94) as described herein. When investigating other organisms the definition encompasses Hsp90 homologues from other species. For instance, Hsp82 from yeast; or HtpG from E. coli.

By “binding partner” we mean a protein, or regions of protein, identified as one half of a pair in A-E above cloned in frame with either an Activation Domain or a DNA Binding Domain that will allow transcriptional activation of a reporter gene when combined with a protein, or regions of protein, identified as the other half of a pair in A-E above cloned in frame with the other of an Activation Domain or a DNA Binding Domain in a yeast two-hybrid assay as described herein.

By “targeting protein” in strain A, we mean any co-chaperone that will define the specificity of the Hsp90 interaction with a client protein. For example, Cdc37 which specifies a kinase client or an immunophilin (such as FkBP51 or FKBP52) which specifies a steroid receptor. The targeting protein may also be Hop or other TPR domain protein needed for the formation of the Hsp90 complex and the client may be Cdk4, topoisomerase I, topoisomerase II, Apaf-1, MAS70 or PP5.

By a “client protein” we mean any protein bound by the Hsp90 complex whereby the Hsp90 complex determines the client proteins fate, whether that be stabilisation, destruction, inhibition or activation.

By “E3 ligase” in strain C, we mean any E3 ligase that interacts with Hsp90 and targets an Hsp90 client for destruction, such as CHIP.

By “Hsp90 inhibitor”, we mean anything that will reduce Hsp90 activity, such as (i) a compound that may bind to Hsp90 and inhibit its activity (e.g. competitive inhibitors or allosteric inhibitors); (ii) a compound, which prevents the transcription, translation or expression of Hsp90 (e.g. ribozymes or antisense DNA molecules); (iii) a compound, which inhibits release of Hsp90 from intracellular stores; and/or (iv) a compound which increases the rate of degradation of Hsp90.

By “Hsp90 agonist”, we mean anything that will increase Hsp90 activity, such as (i) a compound that may bind to Hsp90 and increases, promotes or augments its activity; (ii) a compound, which increases, promotes or augments the transcription, translation or expression of Hsp90; (iii) a compound, which increases, promotes or augments release of Hsp90 from intracellular stores; and/or (iv) a compound which decreases the rate of degradation of Hsp90.

It is preferred that the screening method in accordance with the invention is used for identifying and/or analysing Hsp90 inhibitors comprising the steps of contacting a compound with at least two of yeast strains A-E wherein each yeast strain comprises expression vectors from which a pair of binding partners for a yeast two-hybrid assay are expressed and wherein the binding partner pairs comprise:

A: Hsp90-targeting protein;

B: Hsp90-Hsp90;

C: Hsp90-p23;

D: Hsp90-E3 ligase;

E: Hsp90-Client;

and measuring inhibition of dimerisation between the binding partners.

However, it will be appreciated that the screening method in accordance with the invention is used for identifying and/or analysing Hsp90 agonists.

According to the methods of the invention a group of interactions may be assayed simultaneously. A test compound may inhibit interaction between binding partners in one of the yeast strains and the relative level of inhibition of the different interactions may be analysed to provide valuable information as to the use of the compound as an Hsp90 inhibitor. Alternatively, a test compound may promote interaction between binding partners in one of the yeast strains and the relative level of promotion of the different interactions may be analysed to provide valuable information as to the use of the compound as an Hsp90 inhibitor or agonist.

The methods of the invention are an improvement over known assays of Hsp90 activity because multiple aspects of Hsp90 activity are assayed simultaneously. The assay is also yeast based rather than utilizing mammalian cell lines or purified proteins. Furthermore the methods according to the first aspect of the invention have the advantage that they are capable of providing detailed and specific data relating to the effects of screened compounds on Hsp90 activity. They also have the benefit of identifying lead compounds that would be missed by other screens. Furthermore, compared to prior art screens based on cell lines the method of the first aspect of the invention is also inexpensive (by a factor of 10-100 fold more per test). Furthermore the methods according to the present invention are capable of identifying compounds that inhibit and/or promote Hsp90 activities and have uses outside of the area of cancer therapeutics (e.g. anti-fungal therapeutics).

The methods of the invention offer benefits over known yeast 2-hybrid assays because they do not require identification of an Hsp90 inhibitor or agonist on the basis of inhibition/promotion of binding between single yeast 2-hybrid binding partners. The methods of the invention actually allow identification and/or characterisation of an Hsp90 inhibitor/agonist on the basis of the comparison of the effect a compound has on multiple binding partners.

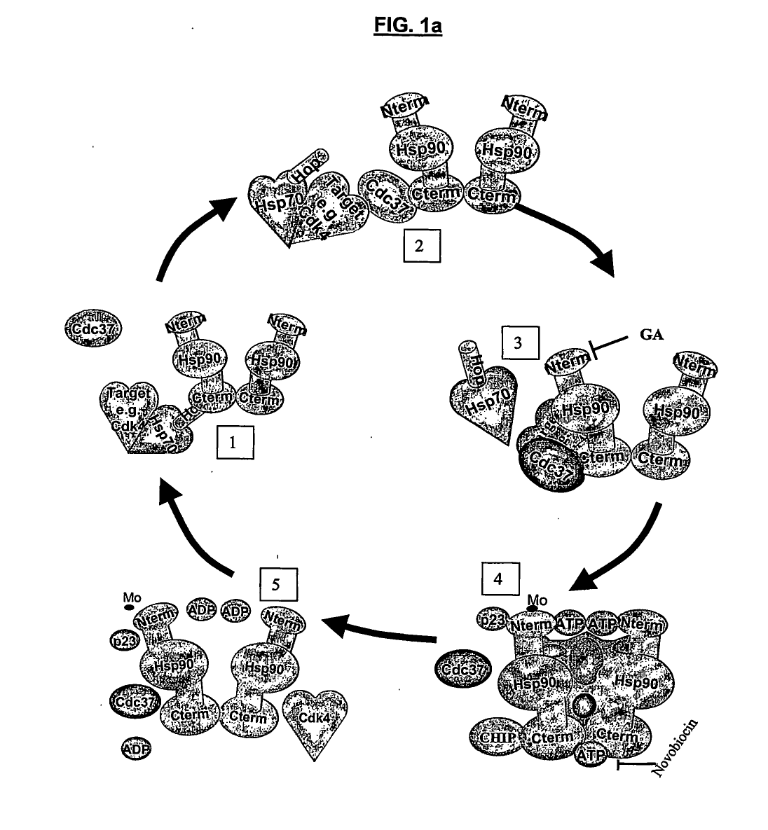

The invention was made following in depth research by the inventors into the interactions of Hsp90 with other molecules. In cell-free systems Hsp90 (as a dimer) can independently bind to and cause folding of proteins. However in vivo Hsp90 does not act independently but functions in combination with co-chaperones. This chaperone complex has far greater specificity than Hsp90 alone due to targeting proteins, such as Cdc37. Client (target) proteins, such as Cdk4, are pre-bound to the Hsp70-Hop-Hip complex. The Hop protein binds to Hsp90 forming a bridge to Hsp70. Subsequent association of the client and targeting proteins to Hsp90 results in dissociation of Hop-Hsp70 from Hsp90. Release of Hop allows the binding of ATP to Hsp90, which in turn allows binding of another Hsp90 cofactor, p23. The binding of ATP results in a conformational change of Hsp90, which causes the client protein to be tightly bound. Release of the client protein requires hydrolysis of ATP to ADP, during which process p23 dissociates from the complex. The subsequent binding of Hop prevents further binding of ATP, GA is also not able to bind to this conformation of Hsp90. Binding of the targeting protein Cdc37 also seems to maintain an inhibition of ATP binding but does not prevent binding of GA. These interactions suggested to the inventors that an Hsp90 cycle exists and they have formulated the cycle shown in FIGS. 1a and 1b.

The inventors believe that variations on the cycle illustrated in FIGS. 1a and 1b are possible. The protein C-terminal HSC70 Interacting Protein (CHIP) can bind in place of the targeting protein, resulting in the proteins bound to Hsp90 being targeted for proteolysis. Also targeting proteins can be specific for different forms of the client protein adding a potential layer or regulation. For example, the immunophilin FKBP51 targets unbound glucocorticoid receptor to Hsp90 while FKBP52 binds to Hsp90 in association with liganded glucocorticoid receptor.

Hsp90 is regulated by the presence or absence of targeting protein. However Hsp90 function is also regulated by additional mechanisms. For instance, the inventors believe that release of v-Src from the chaperone complex may follow serine-threonine phosphorylation of Hsp90 by an unidentified kinase. They also believe that calmodulin binding to Hsp90 regulates the nuclear accumulation of cyclin D1-Cdk4.

The inventors realised, in the light of the Hsp90 cycle discussed above that there are many ways in which Hsp90 activity may be inhibited and/or promoted. For instance, inhibition could block the Hsp90 cycle entirely or just restrict one aspect of Hsp90 activity. In the latter case harmful effects of Hsp90 inhibition could be limited while desirable forms of Hsp90 inhibition could be accentuated. The inventors therefore developed the methods according to the invention to allow the identification of compounds with specified patterns of interaction with the Hsp90 cycle that indicate that such compounds will be putative therapeutic agents for specific clinical indications.

The methods of the invention are preferably based on a yeast two-hybrid system that can evaluate the inhibition or strengthening of interactions of compounds with the Hsp90 cycle. However it will be appreciated that other interaction-trap assays such as mammalian two-hybrid, bacterial two-hybrid or alternatively various types of pull down assay may be developed that fall within the scope of the invention.

The yeast two-hybrid system is based on the discovery that most yeast transcription factors contain separable domains with specialist functions that include a DNA-binding domain (DBD) and an activation domain (AD). It was first shown that fusion of the GAL4 AD with the LexA DBD was sufficient for expression to occur. Initial experiments using the yeast two-hybrid system demonstrated that the fusion of the GAL4-DBD with SNF1 and the GAL4-AD with SNF4 allowed the activation of transcription of the his3 and β-galctosidase reporter genes under the control of the Ga11 promoter in a yeast strain lacking the natural Ga11 activator (Ga14), as the SNF1 and SNF4 proteins bind together and so bring the AD and DBD together. This system has since been extensively used to identify genes for proteins, or regions of proteins, that when cloned in frame with the AD and DBD will allow transcriptional activation of reporter genes. This is widely taken as evidence that the cloned proteins bind to each other (although on occasions this binding can be indirect).

Hence, the two-hybrid system may be used to identify novel binding proteins. A bait protein is fused to the DNA binding domain of a transcription factor (e.g. Ga14). The unknown protein (the prey) is fused to the activation domain and so transcription can be activated if there is an interaction between the bait and prey proteins.

For example, the two-hybrid system has previously been used to study the interactions of Hsp90, Cdk4, Cdc37, Hop, CHIP, FKBP52, FKBP51 and Cyp40. However the methods of the present invention involve a specific combination of two-hybrid sets, each set may be tested and, depending upon the pattern of specific interactions between the compound and the HSP90 binding partner, provides unexpectedly valuable information as to the usefulness of the screened compound for treating particular clinical conditions.

Yeast two-hybrid assays have not previously been used to screen and/or detect Hsp90 inhibitors and/or Hsp90 agonists. Furthermore the use of the two-hybrid system, according to the methods of the invention, to investigate multiple interactions of the same protein with the same compound with a view to comparative levels of inhibition is new to the art. The conventional yeast two-hybrid assays only tests whether a compound interferes with a given interaction and that it is specific to the extent that the compound does not have a particular unrelated effect (i.e. it does not affect growth of yeast or an unrelated interaction). The methods according to the present invention test which of a group of related interactions is most affected by a compound. As Hsp90 is multifunctional, and successful therapy will rely on inhibiting some but not all of these functions, this has clear advantages over known tests.

The screen according to the invention will identify test compounds, which interact with either or both of the binding partners of the yeast two-hybrid system, i.e. compounds that interact directly or indirectly with both the prey and the bait proteins.

Full length Hsp90 protein may be used as a binding partner according to the methods of the invention. However, the inventors have found that Hsp90 fused at its N-terminal with a transcriptional activation domain will not interact with Cdk4, Raf1, Apaf-1 or any other client protein. Fusion of a DNA binding domain to the N-terminal of Hsp90 was also impractical to use in a conventional manner, as there was too high a level of background activation. The inventors have identified that fusion of a DNA binding domain to the C-terminal of Hsp90 overcomes these problems and allows conventional use of the Two-Hybrid system. We have also made the observation that an N-terminal fusion of full length Hsp90 to the transcriptional activation domain of Ga14 inhibits the basal transcriptional activation of the Ga14 DNA binding domain fused to the N-terminal of Hsp90. Furthermore, GA restores this basal level of expression. Accordingly dimerisation may be tested according to the methods of the invention with full length constructs or fragments of Hsp90 and preferably the two-hybrid binding partner comprises a full length or fragment of Hsp90 fusion with the DNA binding domain fused to its C-terminal.

It is preferred that the methods of the invention include a further yeast strain F that comprises the empty expression vectors (i.e. vectors without the constructs coding the 2-hybrid binding partners). It will be appreciated that Strain F constitutes a useful control.

The methods according to the present invention may be advantageously employed to identify Hsp90 inhibitors and/or characterise such inhibitors when a combination of any two of the interactions A-E is used in a comparative assay. In addition, the methods according to the invention may be employed to identify Hsp90 agonists and/or characterise such agonists when a combination of any two the interactions A-E is used in a comparative assay. Such assays may be conducted with or without inclusion of the empty vector control (strain F). However, it will be appreciated that the methods are of great value when more than two yeast strains are employed (e.g. each of strains A-E).

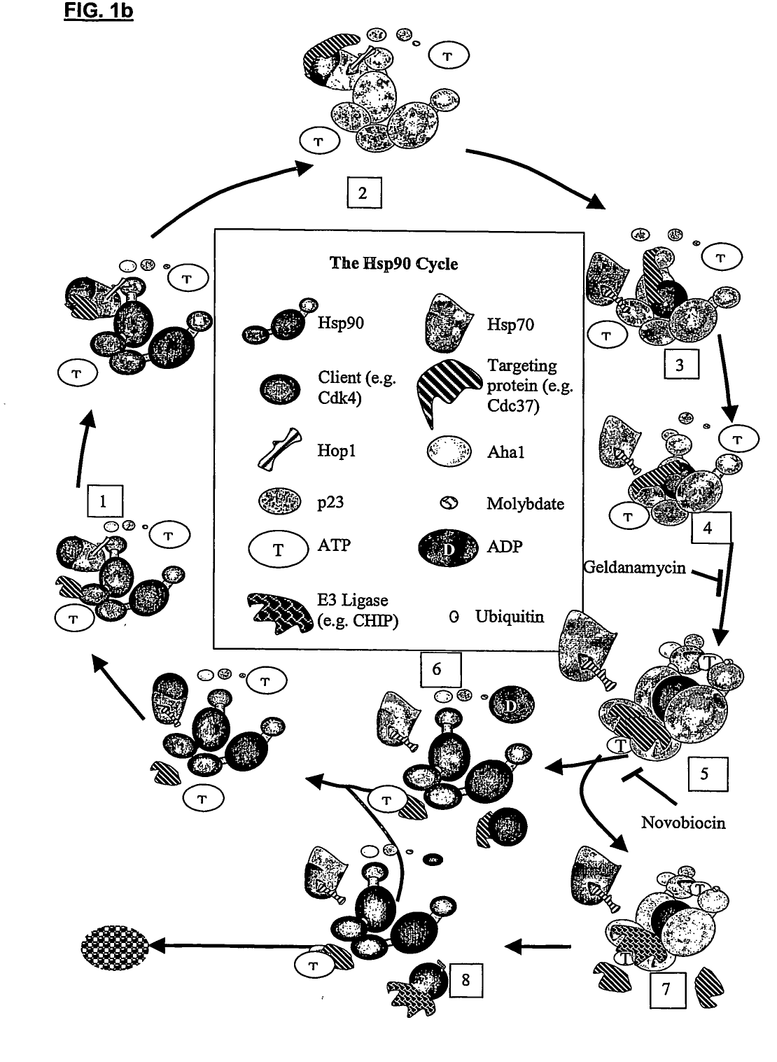

Yeast have many advantages in development of screening systems or biosensors-compared to mammalian cells they are robust; easy and cheap to grow; genetically stable; and represent a reduced health risk to workers (viral contamination of cell lines becomes an issue in low cost high throughput screening). Yeast also have the advantage over bacteria that they are eukaryotes and, as shown in FIG. 2, yeast proteins can substitute for human proteins to make a screen more relevant to a human clinical setting. However, yeast are not always permeable to compounds that would be able to enter mammalian cells. While this is not a problem with known Hsp90 inhibitors (the inventors have found that GA, Macbecin, Herbimycin, 17-AAG, Radicicol and Novobiocin all enter yeast cells easily) it is a potential problem in identifying novel Hsp90 inhibitors and/or Hsp90 agonists with different structures. This problem has been overcome in the past by using yeast mutants that have enhanced sensitivity to toxic chemicals. These mutations are in genes described as Pleiotrophic Drug Resistance (PDR) or Multiple Drug Resistance (MDR) gene. One class of PDR genes are those affecting the ergosterol balance in the yeast membrane. Mammalian cells have cholesterol in the plasma membrane while yeast have ergosterol. Mutations in the gene ERG6 (ISE1) cause cells to become sensitive to a range of lipophilic drugs. Another class of mutations affect active drug uptake or efflux, for example mutations to the ATP dependent efflux pump PDR5 increase drug sensitivity. A combination of mutations from these different pathways further increases sensitivity. In addition to increasing permeability of the plasma membrane and reducing efflux better drug entry can also be brought about by changing the cell wall structure, this can also increase osmotic sensitivity, which facilitates release of proteins. Mutation of the GDP-mannose phosphorylase (SRB1) interferes with the glycosylation of cell wall proteins, this increases drug and osmotic sensitivity. Hsp90 itself is also involved in the maintenance of cell wall integrity via its functional interaction with Ste11. Ste11 is a MAPKKK that normally activates the MAPKK Pbs2 which in turn activates the Hog-1 transcriptional activator. Hog-1 activation modifies the cell wall in response to osmotic shock, disruption of Hog-1 causes drug and osmotic sensitivity. There are also dominant mutations which increase sensitivity to cytotoxic agents, such as ISE2 which increases sensitivity to a range of topoisomerase II inhibitors.

It will therefore be appreciated that the above mentioned yeast represent preferred yeast for use according to the invention. Such PDR mutations increase the range of compounds that may be detected with 2-hybrid systems according to the invention.

A preferred yeast strain has a deletion of the Hog-1 gene. As described above this mutation increases drug sensitivity. Furthermore the Hog pathway is sensitive to Hsp90 inhibition. Inhibition of this pathway is the most significant mechanism of toxicity by Hsp90 inhibitors in yeast. A test compound may inhibit binding in only one yeast strain according to the invention and leave binding in others unchanged. By making all strains Hog-1 negative, inhibition of this pathway becomes irrelevant.

Another preferred yeast strain has a deletion of the STE11 gene. Such strains have similar uses as Hog-1 mutants. Use of Hog-1 deletant strains and STE11 deletants are also advantageous for use with the method of the second aspect of the invention for measuring promoters of dimerisation between the binding partners.

It is preferred that Strain A comprises Hsp90 and a TPR domain protein as yeast 2-hybrid binding partners. The TPR domain protein may be Hop; an immunophilin targeting protein; or an activatable protein (e.g. MAS70 or PP5).

The yeast strains used in a particular screen will depend upon the type of Hsp90 inhibitor and/or Hsp90 agonist that is of interest. Accordingly specific binding partners in strains A-E will be chosen to define a specific aspect of the Hsp90 cycle.

It will be appreciated that the methods of the invention may be adapted so that there are more than 5 test stains A-E. Furthermore a number of different strains corresponding to the same type of strain A-E may be included in a screen.

Preferred sets of yeast strains are described in Examples 1-3.

In a further aspect, the invention extends to the use of the screening methods according to the invention to identify a compound that activates an Hsp90 client protein by changing the balance between inhibition of the client by the Hsp90 and other Hsp90 activities. The screening method may be further used to identify a compound that causes a specific degradation of a particular Hsp90 client by changing the balance between protection of the client by Hsp90. Furthermore, the method may be used to identify a compound that causes targeted degradation of a particular Hsp90 client by changing the balance between Hsp90 binding of targeting and E3 ligase co-chaperones and other Hsp90 activities.

The invention further extends to the use of the screening method according to the invention to identify a compound that causes the inhibition of a particular Hsp90 client by changing the balance between Hsp90 binding to the client and other Hsp90 activities. In addition, the screening method may be used to identify a compound that causes inhibition of another protein and also changes the balance between Hsp90 binding to client proteins and co-chaperones.

A preferred method according to the invention may include the yeast strains illustrated in Table 1.

TABLE 1

| Strain according | | |
|---|---|---|
| to the invention | 2-hybrid constructs | Type of interaction |
| A1 | Hsp90-Cdc37 | Targeting protein |
| A2 | Hsp90-Cdk4 | Client protein |
| A3 | Hsp90-FKBP52 | Targeting protein (TPR domain) |
| B1 | Hsp90-Hsp90 | Dimerisation of Hsp90 |
| B2 | Hsp90-Hsp90 C- | Dimerisation of Hsp90 |
| | terminal | |
| C | Hsp90-p23 | ATP binding modulation |
| D | Hsp90-CHIP | Targeted destruction |
| F | Empty vectors | Control |

According to a preferred embodiment of the invention the method is adapted for high-throughput screening for Hsp90 related interactions for sensitivity to compounds. 96 well plate may be used that test up to 10 compounds at a single concentration for up to 7 different aspects of Hsp90 inhibition. Data produced in the binding assays may be output to spreadsheet and putative Hsp90 inhibitors with particular binding properties may be identified or specific medical indications as illustrated in the specific Examples.

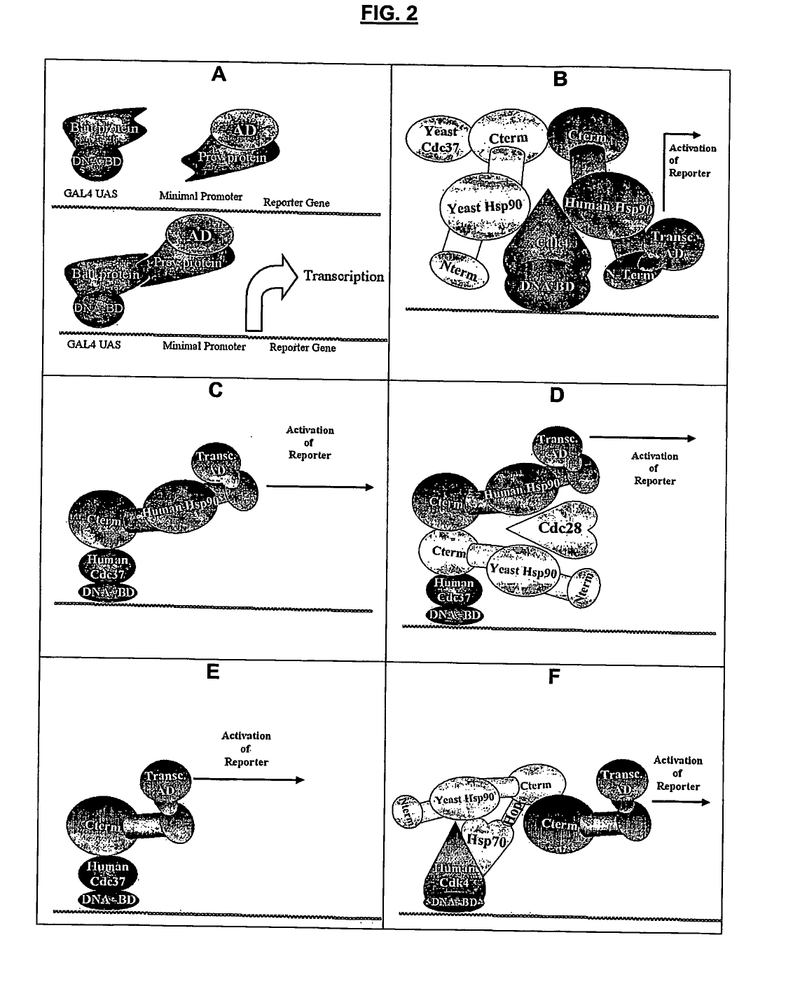

A preferred protocol for carrying but 2-hybrid assays according to the invention involves growth of the yeast strains in media with 1.2M sorbitol (which allows growth of Hog-1 mutant strains—if used) until the yeast reach late exponential phase. An aliquot of each strain is then injected into each well in a single row of a 96 well plate. Strain A in row A etc. (as illustrated in FIG. 3). Each well in column 11 of the plate may contain a known inhibitor of Hsp90, each well in column 12 may contain carrier solvent. The other columns may have single concentrations of test compounds. The plate is incubated for a period of 1 to 24 hours and then centrifuged and the media removed, the cells are snap frozen for subsequent use. A volume of a suitable buffer is added (e.g. 100 mM K₂HPO₄, 100 mM KH₂PO₄, 1 mm DTT) followed by a volume of a chemiluminescent substrate reagent. The plate is then placed in a luminometer and a volume of initiator reagent (for inducing luminescence) injected into each well in the machine. Following a delay of about 20 seconds (post addition of the initiation reagent) an integration time of 5 seconds is used to measure the amount of light given off by each reaction.

The above-mentioned protocol was employed in preliminary experiments conducted using the strains identified in Table 1 a representation of the results is shown in FIG. 3, Strain D actually generates more galactosidase when the interaction is inhibited. The standard Hsp90 inhibitor (geldanamycin) does not inhibit the interaction in strain B. The compound in column 3 is roughly equivalent in the spectrum of Hsp90 inhibition to geldanamycin. The compound in lane 5 is a novel form of Hsp90 inhibitor that only inhibits the interaction in strain B; the compound in column 10 has a broad spectrum of Hsp90 inhibition but does not affect the interaction in strain C or strain D.

The screen according to the invention may be used to identify and/or analyse chemotherapeutic agents. In a preferred embodiment, when using yeast strain A in the screening method, the binding partner pairs comprise Hsp90-FKBP52, and inhibition of binding therebetween is indicative of an inhibitor of prostate cancer.

In a further preferred embodiment, when using yeast strain A in the screening method, the binding partner pairs comprise Hsp90-CDK4, and inhibition of binding therebetween is indicative of an inhibitor of tumors with mutations in the retinoblastoma protein (Rb). In a yet still preferred embodiment, when using yeast strain D, the binding partner pairs comprise Hsp90-CHIP, and inhibition of binding therebetween is indicative of an inhibitor of cystic fibrosis.

The screening method according to the invention may be used to identify and/or analyse antimicrobial agents. The screening method may be used to identify and/or analyse antifungal or antibacterial agents. For example, when using yeast strain A, the binding partner pairs comprise either Hsp82-CHIP or Hsp82-Cpr6, and inhibition of binding therebetween is indicative of an antifungal agent.

The present invention will now be further described, by way of example, with reference to the accompanying drawings, in which:

FIGS. 1a & 1b are schematics illustrating the Hsp90 Cycle;

FIG. 2 is a schematic illustrating the Two-hybrid system;

FIG. 3 is a photograph of a 96 well plate in which a screening method according to the invention has been conducted;

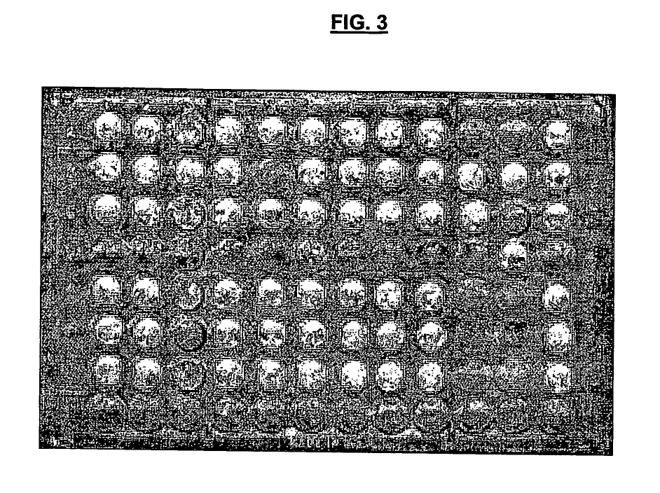

FIG. 4 illustrates the effect of test compounds 1-9 on each of the 2-hybrid constructs in yeast strains in Example 1;

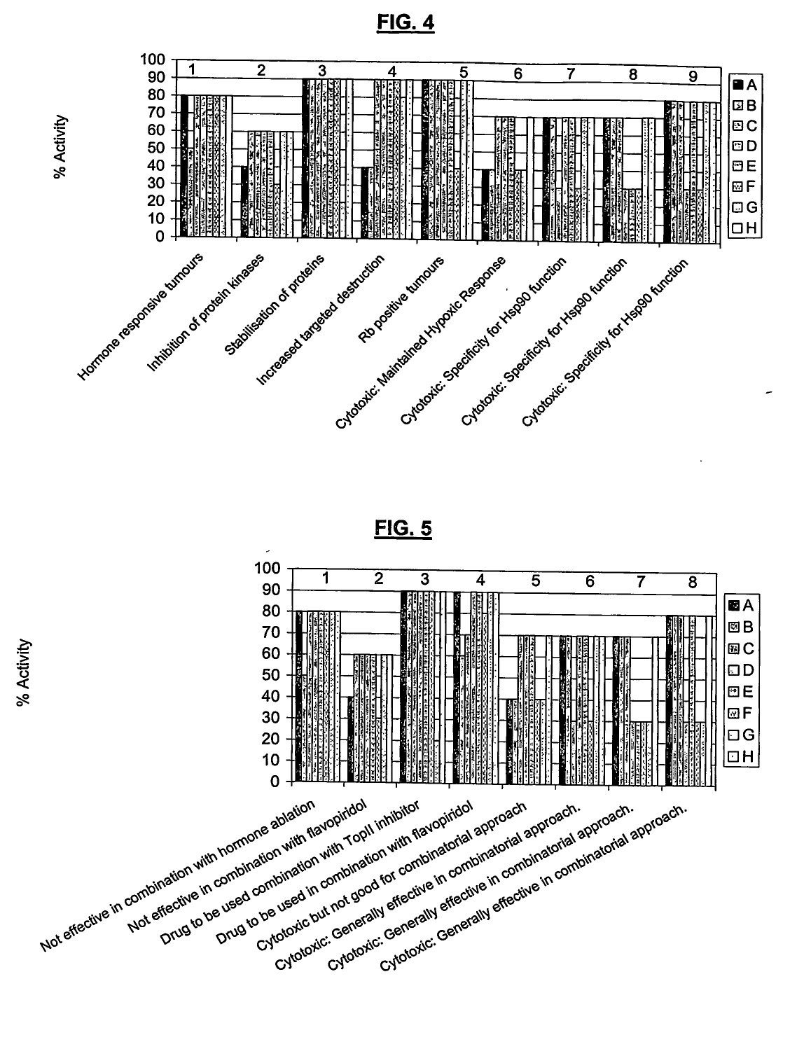

FIG. 5 illustrates the effect of test compounds 1-8 on each of the 2-hybrid constructs in yeast strains in Example 2; and

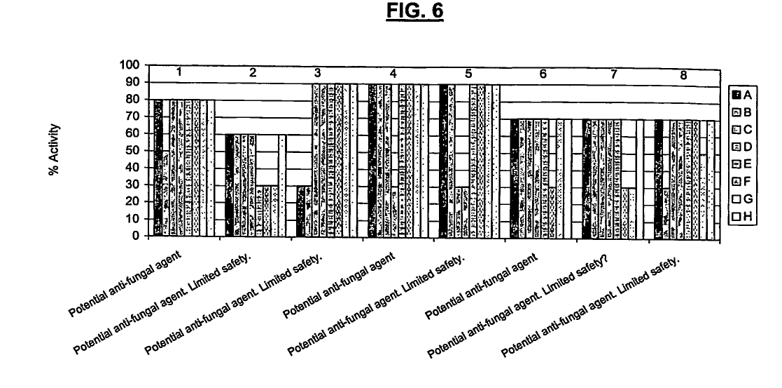

FIG. 6 illustrates the effect of test compounds 1-8 on each of the 2-hybrid constructs in yeast strains in Example 3.

The Hsp90 Cycle is illustrated in FIG. 1a in which 1) Hsp70 binds to and folds proteins. Hsp90 is associated with the complex via the bridging protein Hop. 2) If the protein attached to Hsp70 can be recognised by a targeting protein (e.g. Cdc37) the target protein becomes attached to Hsp90 via the targeting protein. 3) The target protein can then loosely associate with Hsp90. GA can bind at this point blocking the cycle. 4) ATP binding to Hsp90 clamps the target protein into a pocket between the two Hsp90 monomers, maintaining the target protein in an inactive form. This requires the co-cofactor p23 and the ion molybdate. Novobiocin blocks the binding of ATP to the C-terminal of Hsp90, blocking the cycle at this point. 5) Hydrolysis of ATP allows the release of the active target protein.

The Hsp90 Cycle is also illustrated in more detail in FIG. 1b in which [1] Hsp70 binds to and folds proteins, some of which are clients of Hsp90. Hsp90 is associated with the complex via the bridging protein Hop, this association can be at the N-terminal of Hsp90 preventing binding of ATP. [2] If the protein attached to Hsp70 can be recognised by a targeting protein (e.g. Cdc37) the client protein becomes attached to Hsp90 via the targeting protein, Cdc37 can replace Hop binding at the N-terminal of Hsp90 maintaining the inhibition of ATP binding, Hop/Hsp70 can remain associated with Hsp90 at the C-terminal. [3] The client protein can then loosely associate with Hsp90. [4] Binding of the ATP binding modulator Ahal at the N-terminus will destabilise binding of Cdc37 to the N-terminal ATP binding site, allowing binding of ATP. [5] ATP binding to Hsp90 clamps the client protein into a pocket between the two Hsp90 monomers, maintaining the client protein in an inactive form. Binding of ATP is stabilised by binding of another ATP binding modulator, p23, and also requires the ion molybdate. GA can bind at this point blocking binding of ATP and hence the cycle. Novobiocin blocks the binding of ATP to the C-terminal of Hsp90, blocking the cycle at this point [6] Hydrolysis of ATP allows the release of the active client protein. [7] As an alternative to release of active protein, Hsp90 can target its client for destruction, this involves the binding of an E3 ligase (e.g. CHIP) to Hsp90 in place of the targeting protein. [8] Hydrolysis of ATP under these circumstances leads to ubiquitination of the client and its eventual destruction by the proteasome.

FIG. 2 graphically illustrates a two-hybrid system. The basic two-hybrid system (A) was designed for the identification of novel binding proteins. A bait protein is fused to the DNA binding domain of a transcription factor (e.g. Ga14). The unknown protein (the prey) is fused to the activation domain and so transcription can be activated if there is an interaction. In the system according to the invention the hybrid will be expressed alongside the indigenous yeast Hsp90 and co-chaperones. For Hsp90-client interactions the presence of these yeast proteins is required (as shown in B). Interactions with targeting proteins will not require the yeast proteins (as shown in C) but may be influenced by these proteins (as shown in D). Interactions with co-chaperones or targeting proteins do not require the full length Hsp90 (as shown in E). Interactions may also be brought about via yeast Hsp90 and other elements of the yeast Hsp90 complex (as shown in F).

FIG. 3 illustrates the results of a screen using the yeast strains identified in Table 1.

### EXAMPLE 1

This Example illustrates the use of the methods according to the invention to identify Hsp90 inhibitors for different single use therapeutic modalities on mammalian cells in cancer therapy.

Basis: By investigating multiple aspects of Hsp90 action simultaneously the output from the methods of the invention provides mechanistic information as well as identifying Hsp90 inhibitors. In this example 9 compounds are tested with the strains described below. The objective being the identification of an agent that will target mammalian cells so as to kill particular types of cancer cells with the minimum collateral affect on other cells, or to prevent the targeted degradation of mutant CFTR protein while having the minimum affect on other proteins.

1.1 Methods

Strains A-H were developed comprising 2-hybrid constructs as specified below.

| Strain in | Strain according | 2-hybrid | |
|---|---|---|---|
| Figure | to the invention | constructs | Type of interaction |
| A | A | Hsp90-Cdc37 | Targeting protein |
| B | A | Hsp90-FKBP52 | Targeting protein |
| | | | (TPR domain) |
| C | C | Hsp90-p23 | ATP binding |
| | | | modulation |
| D | B | Hsp90-Hsp90 | Dimerisation of Hsp90 |
| E | B | Hsp90-Hsp90 | Dimerisation of Hsp90 |
| | | C-terminal | |
| F | A | Hsp90-Cdk4 | Client protein |
| G | D | Hsp90-CHIP | Targeted destruction |
| H | F | Empty vectors | Control |

The yeast may be grown in a Erlenmeyer flask in media with 1.2M sorbitol (which allows growth of Hog-1 mutant strains) until the yeast reach late exponential phase. 100 μl of each strain is then injected robotically into each well in a single row of a 96 well plate. Strain A in row A etc. (as illustrated in FIG. 3) Each well in column 11 of the plate may contain a known inhibitor of Hsp90, each well in column 12 may contain carrier solvent. The other columns have single concentrations of test compounds.

The plate is then centrifuged and the media removed, the cells are snap frozen on a liquid nitrogen bath for subsequent use. 50 μl of buffer is added (100 mM K₂HPO₄, 100 mM KH₂PO₄, 1 mM DTI) followed by 100 μl of a chemiluminescent substrate reagent (Roche Cat No. 1758241). The plate is then placed in a Lumistar Luminometer, BMG labsystems, Germany and 50 μl of Initiator reagent (Roche Cat No. 1758241) injected into each well in the machine. Following a delay of 20 seconds post addition of the initiation reagent, an integration time of 5 seconds is used to measure the amount of light given off by each reaction.

Results were normalised by measuring β-Galactosidase activity in yeast strains before and after treatment with test compounds. In most assays the level of β-Galactosidase decreases with treatment. Where activity decreases percentage activity is the level of β-Galactosidase activity in the well with treatment divided by the level of activity in the well with no treatment (expressed as a percentage). In some assays level of β-Galactosidase increases with treatment, in these cases percentage activity is the level of β-Galactosidase activity in the well without treatment divided by the level of activity in the well with treatment (expressed as a percentage).

1.2 Results

FIG. 4 illustrates the effect of test compounds 1-9 on each of the 2-hybrid constructs in yeast strains A-H above.

Compound 1 is of interest for treatment of hormone responsive tumours. Inhibition of Hsp90 by this drug would be predicted to have the same effect as testosterone ablation in patients with testosterone responsive prostate cancer. As this drug only affects the targeting protein for glucocorticoid receptors and not any of the other tested aspects of the Hsp90 cycle it would be predicted that it would have reduced harmful affects on normal cells which are not testosterone dependent. This drug would not be predicted to be effective against androgen independent tumours.

Compound 2 would be expected to target any tumour in which protein kinases such as Cdk4 or Src are involved. This drug would be expected to have more general effects than Compound 1 on non-malignant cells but would have less harmful effects than a general Hsp90 inhibitor, as it does not appear to inhibit other aspects of Hsp90 activity (it does not inhibit dimerisation or binding of other targeting proteins such as the immunophilin).

Compound 3 only affects the E3 ligase CHIP whereas Compound 4 affects binding of proteins to the C-terminal (targeting) region of Hsp90. In a majority of cystic fibrosis patients the CFTR protein, although functional, is degraded in an Hsp90 dependent manner. As Compounds 3 and 4 will interfere with the balance between targeted destruction and release but do not inhibit other essential aspects of Hsp90 function either of the drugs may be effective in palliative treatment of CF, in the first case because Compound 3 might allow folding of mutant and wild-type CFTR protein (as well as other proteins), so even mutant CFTR may reach maturity reducing the impact of the mutation. In the case of Compound 4 less aggregate of mutant CFTR might be observed.

Compound 5 inhibits binding of CDK4 to Hsp90 without inhibiting the binding of the protein-kinase targeting proteins Cdc37. It therefore will inhibit cancer cells which require continuous CDK4 activity, but will have reduced impact on non-dividing normal cells. Although, this means Compound 5 is of less value in the treatment of patients with Cdk4 independent tumours (such as those tumours with mutations in the retinoblastoma protein (Rb) it will be potentially more effective than Compound 2 in the treatment of patients with tumours carrying wildtype Rb.

Compound 6 is a general inhibitor of Hsp90 but notably it does not prevent the dimerisation of Hsp90, nor the binding of p23. The fact that CHIP binding is inhibited suggests that a protein targeted to Hsp90 will not be targeted for destruction but could be released in mature form. Hsp90 targeting of Hif-1 for destruction is an important inhibitor of the hypoxic response, although, complete inhibition of Hsp90 binding also prevents this response. Compound 6 as an incomplete inhibitor of Hsp90, which does prevent targeted destruction is potentially an agent that could be used in combination with hypoxia dependent agents.

Compounds 7, 8 and 9 are all general but incomplete inhibitors of Hsp90 activity. In each case the method of the invention allows the nature of this inhibition to be determined.

### EXAMPLE 2

Example 2 illustrates the use of the methods according to the invention to identify Hsp90 inhibitors for different combinatorial therapeutic modalities against cancer cells.

Basis: The inventors have observed that in a number of cases low level (sub-lethal) Hsp90 inhibition sensitises cells to other agents. The mechanism for this activity varies according to the nature of the second agent. In some cases the Hsp90 inhibition simply lowers the threshold of an apoptotic stimulus while in others Hsp90 facilitates the apoptotic stimulus of the second agent. In this example 8 drugs are tested to determine if they would be suitable for this form of combinatorial therapy.

2.1 Methods

The methods of Example 1 were followed except strains A-H were developed comprising 2-hybrid constructs as specified below.

| Strain in | Strain according | 2-hybrid | |
|---|---|---|---|
| Figure | to the invention | constructs | Type of interaction |
| A | A | Hsp90-Cdc37 | Targeting protein |
| B | A | Hsp90-FKBP52 | Targeting protein |
| | | | (TPR domain) |
| C | C | Hsp90-p23 | ATP binding |
| D | B | Hsp90-Hsp90 | Dimerisation of Hsp90 |
| E | B | Hsp90-Hsp90 C- | Dimerisation of Hsp90 |
| | | terminal | |
| F | A | Hsp90-Cdk4 | Client protein |
| G | A | Hsp90- | Client protein |
| | | Topoisomerase II | |
| H | F | empty vectors | Control |

2.2 Results

FIG. 5 illustrates the effect of test compounds 1-8 on each of the 2-hybrid constructs in yeast strains A-H above.

Compound 1 prevents binding of Hsp90 to steroid receptors and would be predicted to have an activity against androgen dependent prostate cancer as described in Example 1. However, it would not be predicted to have any synergistic affect with testosterone ablation. Although, suitable for use instead of this therapy it is not a promising lead for combination therapy.

Compound 2 would also be predicted to prevent binding of Cdk4 to Hsp90. Flavopiridol is a Cdk4 inhibitor and would have a similar affect to Compound 2 but a synergistic effect is unlikely.

In contrast topoisomerase II poisons require the activity of topoisomerase to have maximum effect. Hsp90 inhibition increases sensitivity to these drugs by causing release of topoisomerase II from Hsp90 (where it is held inactive) allowing it to bind to DNA where the action of the drug on the topoisomerase will result in double stranded DNA breaks. Compound 3 will have this effect but does not appear to have other inhibitory effects on Hsp90, it would therefore be a suitable lead compound to develop for combinatorial therapy.

Topoisomerase inhibitors fall into broadly two functional classes poisons as described above and catalytic inhibitors. Catalytic inhibitors such as merbarone and piperazinediones (eg ICRF 187) have more of an effect with decreasing amount of available topoisomerase. Therefore for combination (or mono) therapy involving topoisomeras catalytic inhibitors a compound or compounds should act on Hsp90 reducing the interaction between Hsp90 and topoisomerase in such a way that the topoisomerase activity is reduced.

For combinatorial therapy with Cdk4 inhibitors the ideal drug will have a significant inhibitory effect on Hsp90 but minimal effect on Hsp90 interaction with Cdk4 specifically. Compound 4 is such a drug, it does not inhibit binding of Cdc37 to Hsp90 at all and only has a marginal effect of Cdk4 binding, but it does the binding of p23 and FKBP52 to Hsp90 and therefore would potentially reduce the threshold for activation of apoptosis.

General inhibitors of Hsp90 have already been shown to be effective in this combinatorial approach. For development, a drug should either be very broad range or very specific. Compound 5 has a broad range but will allow some undefined level of Hsp90 activity, this is therefore a less attractive drug for development for this form of combination therapy.

Compound 6 although having a general effect via inhibition of p23 binding does have a level of specificity for Cdk4 and so may be of interest in some form of this combination approach, for example with the drug TPA.

Compounds 7 and 8 are general inhibitors and are therefore of interest as described above.

In this example the client protein can be Topoisomerase I or Topoisomerase II where release of the protein is expected to be of most therapeutic benefit in this context or DHFR where destruction is expected to be of most therapeutic benefit. If the topoisomerase is degraded, then a catalytic inhibitor could be used.

### EXAMPLE 3

Example 3 illustrates the use of the methods according to the invention to identify Hsp90 inhibitors for anti-microbial activity.

Basis: Hsp90 inhibitors have been used as anti-fungal and anti-bacterial agents, they have high activity against protozoal pathogens. However, their use is limited by the toxicity of these agents with mammalian cells. In this example a specific set of yeast strains are chosen in order to identify Hsp90 inhibitors that will only target fungal proteins.

3.1 Methods

The methods of Example 1 were followed except strains A-H were developed comprising 2-hybrid constructs as specified below.

| Strain in | Strain according | 2-hybrid | |
|---|---|---|---|
| figure | to the invention | constructs | Type of interaction |
| A | A | Hsp90-Cdc37 | Targeting protein |
| | | | human |
| B | A | Hsp82-fungal | Targeting protein fungi |
| | | Cdc37 | |
| C | A | Hsp90-FKBP52 | Targeting protein |
| | | | (TPR domain) |
| | | | human |
| D | A | Hsp82-Cpr6 | Targeting protein |
| | | | (TPR domain) |
| | | | yeast |
| E | B | Hsp90-Hsp90 | Dimerisation of |
| | | C-terminal | Hsp90 human |
| F | B | Hsp82-Hsp82 | Dimerisation of |
| | | C-terminal | Hsp90 yeast |
| G | A | Hsp90- | Client protein human |
| | | Topoisomerase | |
| | | II | |
| H | F | empty vectors | Control |

2.2 Results

FIG. 6 illustrates the effect of test compounds 1-8 on each of the 2-hybrid constructs in yeast strains A-H above.

Compound 1 is a potential antifungal agent as it will inhibit the interaction of fungal Cdc37 with the yeast Hsp90 (Hsp82) but will not inhibit the interaction of human Cdc37 with Hsp90.

Compound 2 is a potential anti-fungal agent as it inhibits yeast Hsp82 dimerisation, but it also has an effect on human Hsp90 dimerisation and is therefore likely to be toxic to human cells. Similarly with Compound 3 the interactions of both human and yeast Cdc37 with Hsp90.

Compound 4 is a potential anti-fungal agent because it interferes with the interaction of the yeast immunophilin Cpr6 with Hsp90 but does not interfere with the interaction of the human immunophilin FKBP52 with Hsp90. While Compound 5 is of limited use as it is a more general inhibitor of immunophilin interactions with Hsp90.

Compound 6 is a potentially useful agent as it just inhibits dimerisation of the yeast Hsp82.

Compound 7 effects yeast Hsp82 dimerisation and not human Hsp90 dimerisation, but in this assay it also effects the interaction of human Hsp90 with a client protein. This may because the drug has inhibited the interaction of recombinant human Hsp90 with indigenous yeast Hsp82. Compound 8 effects the interaction of yeast Cdc37 but not human Cdc37 with Hsp90. However, it also effects the interaction of Hsp90 with the human client protein topoisomerase II. This may be because of the interference of the interaction of a yeast targeting protein topoisomerase II or with Hsp90. This drug would also require further analysis.

In place of Hsp82 and yeast Cdc37 we can use the E. coli homologue of Hsp90 (HtpG) and any appropriate client protein, enabling us to identify an agent that inhibits the bacterial cells but does not damage mammalian cells.

1. A screening method for identifying and/or analysing Hsp90 inhibitors and/or Hsp90 agonists comprising the steps of:
contacting a compound with at least two of yeast strains A-E wherein each yeast strain comprises expression vectors from which a pair of binding partners for a yeast two-hybrid assay are expressed and wherein the binding partner pairs comprise:
A: Hsp90-targeting protein;
B: Hsp90-Hsp90;
C: Hsp90-p23;
D: Hsp90-E3 ligase; and
E: Hsp90-Client, and
measuring inhibition and/or promotion of dimerisation between the binding partners.

2. The screening method according to claim 1 wherein the compound is contacted with more than two yeast strains A-E.

3. The screening method according to claim 2 wherein the compound is contacted with each yeast strain A-E.

4. The screening method according to claim 1 wherein the compound is contacted with a further yeast strain F comprising empty expression vectors.

5. The screening method according to claim 1 wherein Hsp90 binding partners comprise a full length or fragment of Hsp90 with a DNA binding domain fused to its C-terminal.

6. The screening method according to claim 1 wherein the yeast strains are engineered to have increased permeability or sensitivity to compounds.

7. The screening method according to claim 6 wherein the yeast strain has a deletion of the Hog-1 gene.

8. The screening method according to claim 6 wherein the yeast strain has a deletion of the STEII gene.

9. The screening method according to claim 1 wherein yeast strain A comprises Hsp90 and a targeting protein selected from the group consisting of Hop, an immunophilin targeting protein, Cdk4, topoisomerase II, Cdc37, Apaf-1, FKBP52, MAS70 and PP5 as binding partners.

10. The screening method according to claim 1 wherein yeast strain B comprises Hsp90 and CHIP as binding partners.

11. The screening method according to claim 1, wherein the screen is used to identify and/or analyse chemotherapeutic agents.

12. The screening method according to claim 11, wherein when using yeast strain A, the binding partner pairs comprise Hsp90-FKBP52, and wherein inhibition of binding therebetween is indicative of an inhibitor of prostate cancer.

13. The screening method according to claim 11, wherein when using yeast strain A, the binding partner pairs comprise Hsp90-CDK4, and wherein inhibition of binding therebetween is indicative of an inhibitor of tumors with mutations in the retinoblastoma protein (Rb).

14. The screening method according to claim 1, wherein when using yeast strain D, the binding partner pairs comprise Hsp90-CHIP, and wherein inhibition of binding therebetween is indicative of an inhibitor of cystic fibrosis.

15. The screening method according to claim 1, wherein the screen is used to identify and/or analyse antimicrobial agents.

16. The screening method according to claim 15, wherein the screen is used to identify and/or analyse antifungal or antibacterial agents.

17. The screening method according to claim 16, wherein when using yeast strain A, the binding partner pairs comprise either Hsp82-CHIP or Hsp82-Cpr6, and wherein inhibition of binding therebetween is indicative of an antifungal agent.

18-19. (canceled)

20. A medicament comprising an Hsp90 inhibitor or Hsp90 agonist identified by a process comprising:
contacting a compound with at least two of yeast strains A-E wherein each yeast strain comprises expression vectors from which a pair of binding partners for a yeast two-hybrid assay are expressed and wherein the binding partner pairs comprise:
A: Hsp90-targeting protein;
B: Hsp90-Hsp90;
C: Hsp90-p23;
D: Hsp90-E3 ligase; and
E: Hsp90-Client,
measuring inhibition and/or promotion of dimerisation between the binding partners; and
identifying an Hsp90 inhibitor or Hsp90 agonist.

21. The composition of claim 20, wherein the medicament is adapted for use in cancer therapy, chemotherapy, as a chemotherapeutic agent, or as an antimicrobial agent.
